(19) United States
(12) Patent Application Publication
(10) Pub. No.: US 2006/0165802 A1
(43) Pub. Date: Jul. 27, 2006

(54) Medium containing water with increased viscosity, method for production and use thereof

(76) Inventors: Thomas Lotzbeyer, Eching (DE); Michael Volland, Karlsfeld (DE); Eva Wittmann, Traunreut (DE)

Correspondence Address: FULBRIGHT & JAWORSKI, LLP, 666 FIFTH AVE, NEW YORK, NY, 10103-3198, US

(21) Appl. No.: 10/528,710

(22) PCT Filed: Sep. 23, 2003
(86) PCT No.: PCT/EP03/10570

§ 371 Date: Apr. 25, 2005

(30) Foreign Application Priority Data
Sep. 23, 2002 (DE) 10244124.3

(51) Int. Cl. C12P 19/04; A61K 9/14

(52) U.S. Cl. 424/488; 435/101

(57) ABSTRACT

The invention concerns a water-containing medium with an increased viscosity containing a gellable polymer component with phenolic substituents modified with the aid of oxidases, characterized in that it was modified by a) a protein with polyphenol oxidase activity and/or b) an enzyme mixture containing hydrolases, oxidoreductases and peroxidases. The medium modified in this manner can be a gel that is preferably in a (partially) dried or (partially) rehydrated state, whose viscosity or gel strength can be selectively adjusted and which, even after a drying process and rehydration, again stably attains the original viscosity or gel strength. In addition the water-containing medium does not have any by-products that could have an adverse effect on quality of the gel or its sensory properties. In addition to the water-containing medium, a process is also proposed that can be used to produce the corresponding media with increased viscosity and which relies on the use of oxidoreductases, peroxidases, hydrolases and/or catalases. Food industry, cosmetics and pharmaceutical purposes are intended as areas of application for the proposed water-containing media.

The present invention concerns a medium containing water with increased viscosity, a process for its production and the use of the medium.

Texturing systems play an important role as additives especially in the food and cosmetic field and impart the desired consistency to the mixtures to which they are added. Of major importance in this connection is, on the one hand, a selected increase in the viscosity of the product as well as, on the other hand, the formation of gel-like structures.

A gel is formed by the cross-linking of numerous molecules by intermolecular interactions. A three-dimensional network is formed in this process in which relatively large amounts of water can then be bound. Examples of this are the formation of pectin gels in the presence of high sugar concentrations or after addition of Ca²⁺-ions in the manufacture of jams or the formation of κ-carrageenan gels in the presence of K⁺-ions.

Due to their low molecular weight or their inability to form gels by hydrophobic or electrostatic interactions, many polysaccharides such as sugar beet pectin are unsuitable or only poorly suitable for increasing viscosity or for gel formation. In this case new technologies are desirable which could be used to overcome the described limitations.

As has been demonstrated in recent years, numerous polysaccharides of the plant cell wall such as pectin in sugar beet or arabinoxylans in wheat grain, particularly as cinnamic acid derivatives, contain phenolic groups that are usually bound to these polymers by ester bonds. These phenolic groups can react with one another by a chemical or enzyme-catalysed reaction. When a large number of phenolic groups of several polysaccharide molecules bind to one another this can ultimately lead to the formation of a three-dimensional network and thus also to the formation of a gel.

Following this principle and according to U.S. Pat. No. 4,672,034 a sugar beet pectin containing ferulic acid was modified with the aid of an oxidizing system consisting of at least one oxidizing substance and an enzyme for which this oxidizing substance is a substrate and allowed to form a gel. The described systems consist of the oxidizing compound hydrogen peroxide and the enzyme peroxidase.

WO 96/03 440 describes a process for gel formation or for increasing the viscosity of an aqueous solution by adding the enzyme laccase from micro-organisms to form a gellable polymeric substance which has substituents containing phenolic groups. An enzymatic pretreatment to remove some of the arabinose groups from the polymer before the enzymatic cross-linking to improve gel formation is also proposed. It also describes the drying or dehydration of the gel and the use of the dry product as an adsorbent for aqueous liquids.

A method for gel formation or for increasing the viscosity of an aqueous solution with the aid of a gellable polymer containing phenolic groups (e.g. sugar beet pectin) is disclosed in WO 97/27 221. In this case gel formation is based on the addition of an esterase (pectin methyl esterase) and an oxidase (laccase) or peroxidase in the presence of a substance that can be oxidized by this enzyme. Also in this case the drying and use of the dry product is described.

Norsker et al. (Food Hydrocolloids, 14, (2000, p. 237-43) describe a process for the enzymatic gelling of sugar beet pectin in foods such as juices, milk, minced meat with the aid of two different laccases and a peroxidase. However, in foods treated in this manner the enzymatic reactions result in an undesired bleaching of anthocyans and fat oxidations that are undesired for sensory reasons.

In summary it can be ascertained that the processes described in the literature for enzymatic gelling of polysaccharides containing phenols have various limitations especially for applications in cosmetics and for foods:

Peroxidases rely on the addition of an oxidizing substance which is usually hydrogen peroxide for a successful gel formation. In the case of longer reactions this compound results in an undesired hydrolysis of the polysaccharide chains which leads to a destruction of the gel that is formed. Hydrogen peroxide can also cause undesired chemical oxidation of the fats and oils that are often present in cosmetic formulations and formation of rancid-like odours. Moreover the hydrogen peroxide may not be added directly to foods.

Laccases occur in plants and fungi. They are responsible for the synthesis and degradation of lignin present in plants. They do not require addition of an additional oxidizing compound for their reaction since they are able to oxidize phenolic compounds in the presence of oxygen. However, they can only catalyse the oxidation of diphenolic compounds. They cannot convert monophenolic compounds such as tyrosine.

During the course of the laccase-catalysed reaction there is a direct electron transfer from the phenolic compound to be reduced to oxygen. A radical is formed in this process which, in the subsequent step, undergoes a non-enzymatic reaction and thus leads to the cross-linking of the ferulic acid-containing polysaccharides. As already mentioned (Food Hydrocolloids, 14, (2000), p. 237-43) the radical product which is formed enzymatically may react with lipids or unsaturated fatty acids may be oxidized in foods and cosmetic substances during the course of the reaction which then results in the already mentioned undesired rancid taste and odour.

Laccases can, however, also be used for enzymatic bleaching. However, in foods and cosmetic formulations the associated reaction often causes a loss of colouring which is undesirable.

According to the state of the art the enzymes which are used in each case to cross-link the polysaccharides remain in an active form in the end-product. As a consequence it is very difficult or impossible to influence the gel strength and formation of the described undesired by-products.

Furthermore, the use of the said enzymes in foods is primarily only allowed when they no longer exert any effect in the end-product, i.e. they are no longer present in an active form.

Laccases occur in higher concentrations only in micro-organisms that have to be cultured in order to obtain the enzyme. The subsequent isolation of the enzyme requires considerable effort for extraction and purification by precipitations, chromatographic methods etc.

Furthermore laccases cannot form gels or only form very weak gels with the phenolic groups of the sugar beet pectin especially in the presence of increased protein or salt concentrations.

On the basis of this state of knowledge the object of the present invention was to provide a water-containing medium with an increased viscosity containing a gellable polymer component containing phenolic substituents which has been modified by oxidases, a process for the production of a medium and the use thereof.

In this connection priority was given above all to the control of the enzyme reaction in order to build-up a defined viscosity or to form a gel having a defined gel strength and to avoid formation of undesired by-products in the end-product due to the enzyme.

This object was achieved with a corresponding medium in which the modification occurred by means of
a) a protein having polyphenol oxidase activity and/or
b) an enzyme mixture containing hydrolases, oxidoreductases and peroxidases.

According to the invention “increased viscosity” is understood as a state of the water-containing medium in which the gellable polymers are in a swollen state or in which gel formation has already started, progressed or is completed and in which the water-containing medium is thus in a thickened, viscous, semi-solid or solid state.

It was possible to completely fulfil the objectives using this medium: The increased viscosity can actually be selectively adjusted by the enzymatic modification and kept stable without secondary reactions. Moreover, the media according to the invention have none of the otherwise disadvantageous by-products. In the case of the claimed water-containing media it was completely surprising that the increase in the viscosity in the medium and the associated gel formation can be predetermined so exactly and can be stably maintained at the desired magnitude even over a long period, that these media can now also be used for fields of application that were previously completely foreign to them. Furthermore, the desired original preset gel strength is reproducible: Thus the water-containing medium according to the invention can be readily dried and subsequently rehydrated to form a gel having the original strength even after a long storage period.

The gel-like medium can also be frozen and thawn again without a significant escape of water or destruction of the gel structure occurring. Finally the gel that is formed can be heated to temperatures above 100° C. without there being significant changes in its structure or water binding capacity.

This was unpredictable to such a degree.

Not least for this reason the present invention provides that the medium according to the invention is preferably a gel and particularly preferably a gel in a (partially) dried and/or (partially) rehydrated state.

In contrast to laccases (p-diphenol oxidases; EC 1.10.3.2) polyphenol oxidases have a monophenolase as well as a diphenolase activity. They occur in higher concentrations in plants and are used there among others to defend against micro-organisms by forming polyphenolic compounds.

Hence polyphenol oxidases combine two different enzyme activities: On the one hand, a monophenolase activity which catalyzes the o-hydroxylation of a mono-phenol to form o-diphenol and distinguishes these enzymes from other phenol oxidizing enzymes such as peroxidase or laccase. On the other hand, polyphenol oxidases have a diphenolase activity which catalyzes an oxidation of two o-diphenols to two o-diquinones and the simultaneous reduction of an oxygen molecule.

In contrast to laccases, the reaction step of oxidizing the diphenol does not result in a radical end-product of the enzyme reaction in the case of polyphenol oxidases which is why the undesired secondary reactions known for the laccase and peroxidase reaction such as fat oxidation or bleaching do not occur.

Polyphenol oxidases such as the known tyrosinase (EC 1.14.18.1) occur in higher concentrations in fruits, bulbs or leaves so that in some cases they can also be used without extraction or purification for technological processes e.g. in the case of tea fermentation to produce “black tea”.

Tyrosinases are also found in higher concentrations in many by-products that are formed during food processing and have previously not been utilized. Thus high tyrosinase activities are detected in the fruit water of potatoes, a by-product of potato starch production. It is usually sufficient to separate the starch and fibrous components of the potato to obtain an extract with an adequate tyrosinase activity to enzymatically cross-link sugar beet pectin.

The special advantages of the claimed modification must also be seen in the polyphenol oxidase activity which is why it should be regarded as advantageous when the polymer component carries monophenolic substituents.

In this connection the present invention preferably also provides a medium whose polymer component is at least one polysaccharide and in particular one containing (un-)substituted cinnamic acid ester groups and which preferably contains an arabinoxylan and/or a pectin as a polysaccharide.

The medium according to the present invention exhibits particulary good properties when the pectin component is derived from Chenopodiaceae and in particular from sugar beet or pulps thereof.

However, as a special variant the invention also provides a medium which contains pectin in which at least one of the arabinose groups has been removed which should preferably be carried out under slightly acidic conditions at a pH between 6.0 and 7.5 and/or with the aid of an enzyme for which arabinofuranosidase is preferably provided.

The arabinoxylan component which is also considered as a preferred polysaccharide can be derived within the scope of the present invention in particular from cereals such as maize or wheat and in this regard especially from flour or coarse meal.

With regard to the polymer component, variants are regarded as preferred which have been modified by a polyphenol oxidase and in this case preferably by a tyrosinase.

The polyphenol oxidase that is used for this can in this case be in particular derived from plants of the Solanaceae family, in particular preferably from potatoes as well as from apples, aubergines, chicory, bananas, avocado, tea plants or mushrooms.

As an alternative or in addition to the modification of the polymer component by a protein with polyphenol oxidase activity, the invention considers it to be important to use an enzyme mixture for this purpose. A variant has proved to be particularly suitable in this connection in which the modification is carried out using an enzyme mixture which contains a β-galactosidase, glucose oxidase, peroxidase and/or optionally a catalase.

As already mentioned a major advantage of the water-containing medium is that it can be dried and rehydrated without impairing quality. Hence a medium is also regarded as preferred which has been subjected to a drying process: in this case the drying can of course be carried out at low (freeze-drying) or at elevated temperatures and in both cases also in a vacuum which additionally underlines the advantages.

The invention also provides a medium in which the enzymes contained therein and particularly preferably the enzymes responsible for modification i.e. in particular oxidoreductases, peroxidases and/or hydrolases are in an inactive form after the modification is completed, so that they can no longer have a technological effect.

Furthermore, media have proven to be particularly suitable which contain enzymes that have been chemically and/or thermally inactivated.

When using a tyrosinase as a typical polyphenol oxidase it is for example possible to thermally inactivate the enzyme in a gel for 15 min at a temperature of 95° C.; for a chemical inactivation of the tyrosinase it is sufficient to add a 1% ascorbic acid solution or to add 1% ascorbic acid to the dried gel or to the dried viscous solution.

In addition to the water-containing and specially modified medium itself, the present invention also concerns its use for foods, cosmetics and/or for pharmaceutical purposes, particularly preferably as texturing agents, viscosity-enhancing agents, gelatinizing agents, film formers, as rheological additives or as stabilizers. The medium according to the invention containing inactivated enzymes can thus be added without problems to foods and cosmetic and pharmaceutical products to increase their viscosity or to form a gel-like structure. In particular with these applications the enzyme (mixture) can, in contrast to the previously known variants, no longer lead to the formation of undesired by-products due to the inactivation of the enzyme component.

The present invention also claims a process for producing a water-containing medium having an increased viscosity which is characterized in that
a) at least a portion of a gellable polymer component containing phenolic substituents is dissolved first in an aqueous medium then
b) a partly dissolved oxidoreductase and/or peroxidase and/or hydrolase and/or catalase of plant or fungal origin is added at room temperature to the solution from step a), subsequently
c) the solution from step b) is stirred for at least 15 minutes at temperatures between 15 and 60° C. and finally
d) the enzymes present in the solution obtained from step c) are optionally thermally and/or chemically inactivated.

The combination of the claimed process steps ensures that the medium obtained in this manner likewise has or develops the advantages that have already been presented.

In this connection process step d) optionally provides that the enzyme component is inactivated. All reactions of the added enzyme (mixture) are thus ended. The viscosity of the medium or its gel strength no longer changes and also the otherwise undesired side reactions (bleaching of dyes or enzymatic fat oxidations) caused by the enzyme (mixture) used for gel formation can no longer occur.

It has turned out to be very advantageous when in process step a) at least one member from the series of oligosaccharide- or polysaccharide-, alcohol-, lactate-, glutamate-, pectin-, and a lactose-containing medium, preferably milk or a milk-containing medium is added first as a polymer component.

Thus the use of compounds present in foods is preferably provided which can either form hydrogen peroxide during the course of an enzyme reaction or can be converted into compounds that can be used to form hydrogen peroxide during a further enzyme reaction. The polysaccharides (hydrolases and oxidases), oligosaccharides (hydrolases and oxidases), alcohols (alcohol oxidase and peroxidase), lactate (lactate oxidase and peroxidase), glutamate (glutamate oxidase and peroxidase) that are claimed for this purpose in the medium are available to form oxidizing substances such as those that are used or required to enzymatically form gels of polysaccharides containing phenolic groups and these oxidizing compounds can be used to increase the viscosity or form gels by enzymatically cross-linking the phenol-containing polysaccharides.

Within the scope of the present invention it is also preferred that at least one galactosidase, glucose oxidase, horseradish peroxidase, laccase or polyphenol oxidase is added in process step b).

Lactose is already in some cases removed from milk or milk products with the aid of β-galactosidase since a not inconsiderable proportion of the population suffers from lactose intolerance. However, in the present process the lactose present in the preferred medium milk yields, after conversion by β-galactosidase and glucose oxidase, the hydrogen peroxide required for gel formation with the aid of horseradish peroxidase. Hence the products of this enzyme reaction serve as auxiliary substances for gel formation in the course of the described enzymatic reaction cascade. Hence for the first time the following two effects are achieved with the proposed process: On the one hand the lactose that is often undesired is removed from the food, on the other hand, the desired texturing of the medium is obtained by gel formation.

Finally the solution obtained in this process from process step b) can be subjected to at least one drying step optionally after adding other gellable and optionally modified polymers. The powder obtained from the drying step can then according to the invention be also rehydrated at a later time.

Another subject matter of the present invention is a water-containing medium with an increased viscosity that can be produced by the process described above. A variant is particularly preferred in which the enzymes which it contains and in particular the enzymes added in process step b) are present in a non-activated form.

Like the already claimed water-containing medium itself, any water-containing medium obtained by this process can also be used in foods, in cosmetics and/or for pharmaceutical purposes, particularly preferably as texturing agents, viscosity enhancing agents, gelatinizers, as film formers, as rheological additives or as stabilizers.

In summary it may be ascertained that the water-containing medium with an increased viscosity according to the invention has particular advantages due to the fact that its viscosity or gel strength can be selectively adjusted, that the resulting viscosity or gel strength is stable and durable and that the medium again builds up the original viscosity or gel strength in a stable and reproducible manner even after prior drying and rehydration. Furthermore, the medium according to the invention is free of undesired by-products because the enzymes used for modification or all other enzymes that are present naturally or are added artificially can be inactivated and the respective inactivation step has no adverse effect whatsoever on the viscosity or gel strength of the medium.

With regard to applications in the food or cosmetic field where the influence of the matrix i.e. the ingredients present in the product such as proteins, salts, fats or sugars are also of major importance for gel formation it has turned out that the gels formed using polyphenol oxidases especially in the presence of higher salt and protein concentrations are much better than comparable gels and those obtained with the aid of laccases.

The following examples elucidate the distinct advantages of the present invention.

### EXAMPLES

### Example 1

Cross-Linking the Medium with Tyrosinase and Drying and Rehydrating the Powder that is Obtained

0.75 g Pectin was stirred into 25 ml 60° C. warn water and completely dissolved. Then the pH of the solution was adjusted to pH 6.0 with 0.1 M NaOH and 1 mg tyrosinase (Sigma t-7755) that had been previously dissolved in 1 ml 100 mM H₂NaPO₄ buffer (pH 6.5) was added. The reaction solution was firstly allowed to stand for 3 h in a covered vessel at 50° C. and afterwards for 20 h at 20° C. and subsequently the gel was frozen at −18° C. It was dried at 0.1 hPa in a freeze-drying apparatus. 0.3 g of the dried powder was subsequently dissolved in 10 ml water at 60° C.

A reaction solution without tyrosinase served as a comparison. The gel strength of the various samples with and without tyrosinase was measured with a Texture analyzer TA-XT2 (Stable Microsystems) using a P20 (20 mm) probe and a cylindrical sensor (5 kg aluminium) at a rate of 3 mm/sec and a path of 5 mm. The gels were in crystallizing dishes of 50 mm in diameter. Table 1 shows the dependency of the force F on the enzyme concentration.

TABLE 1
Dependency of gel strength (force F) on the enzyme concentration
(concentration sugar beet pectin 3%; pH 6)

| | | | | |
|---|---|---|---|---|
| enzyme concentration | 0 | 0.4 | 0.7 | 1.0 |
| [mg/25 ml] | (comparison) | | | |
| force [g] | no gel | 8 | 39 | 269 |

### Example 2

Cross-Linking the Medium with Tyrosinase from Potatoes

In order to extract tyrosinase from potatoes, the juice from 1 kg potatoes was extracted (AFK Juice extractor, Aachen), the potato juice was centrifuged at 4000 rpm for 20 min and the supernatant was filtered through a black band filter. 0.75 g sugar beet pectin was dissolved at 50° C. in 25 ml of the filtrate obtained in this manner while stirring. After the gel formation which started after 3 h at 20° C. was completed, the gel was frozen at −18° C. and dried at 0.1 hPa in the freeze-drying apparatus. Finally 0.3 g of the dried powder was dissolved in 10 ml warm water at 60° C.

### Example 3

Cross-Linking the Medium with β-Glucosidase/Glucose Oxidase/Horseradish Peroxidase Mixture and Drying and Rehydrating the Powder that is Obtained

0.75 g Pectin and 3.5 mg lactose was stirred into 25 ml 60° C. warm water and completely dissolved. Then the pH of the solution was adjusted to pH 6.0 with 0.1 M NaOH and 5 μg β-galactosidase (Sigma G-6160), 250 μg glucose oxidase (Sigma G-7016) and 1 mg peroxidase (Sigma P-6782) that had been previously dissolved together in 1 ml 100 mM H₂NaPO₄ buffer (pH 6.5) were added. The reaction solution was then allowed to stand for 1 h at 20° C. and subsequently the resulting gel was frozen at −18° C. It was dried at 0.1 hPa in the freeze-drying apparatus. 0.3 g of the dried powder was subsequently dissolved in 10 ml warm water at 60° C.

### Example 4

Cross-Linking the Medium with Laccase, Thermally Inactivating the Enzyme, and Drying and Rehydrating the Powder that is Obtained (Comparison)

0.375 g Pectin was stirred into 25 ml 60° C. warm water and completely dissolved. Then the pH of the solution was adjusted to pH 5.0 with 0.1 M NaOH and 0.2 mg laccase (ASA Enzyme, Order No. 2020) that had been previously dissolved in 1 ml water was added. The reaction solution was allowed to stand for 24 h at 20° C. and then the laccase was inactivated by heating the gel for 20 min to 90° C. Subsequently the gel was frozen at −18° C. and then it was dried at 0.1 hPa in the freeze-drying apparatus. 0.3 g of the dried powder was subsequently dissolved in 10 ml warm water at 60° C.

### Example 5

Cross-Linking the Medium with Tyrosinase and Thermally Inactivating the Enzyme, and Drying and Rehydrating the Powder that is Obtained

0.75 g Pectin was stirred into 25 ml 60° C. warm water and completely dissolved. Then the pH of the solution was adjusted to pH 6.0 with 0.1 M NaOH and 1.2 mg tyrosinase (Sigma T-7755) that had been previously dissolved in 1 ml 100 mM H₂NaPO₄ buffer (pH 6.5) was added. Subsequently it was allowed to stand in a water-bath and in a covered vessel firstly for at least 2 h at 50° C. and afterwards for 20 h at 20° C. and then the tyrosinase was inactivated by heating the gel for 40 min to 90° C. The gel was then frozen at −18° C. and dried at 0.1 hPa in the freeze-drying apparatus. Finally 0.3 g of the dried powder was subsequently dissolved in 10 ml warm water at 60° C.

### Example 6

Cross-Linking the Medium with Horseradish Peroxidase and Thermally Inactivating the Enzyme, and Drying and Rehydrating the Powder that is Obtained

0.5 g Pectin was stirred into 25 ml 60° C. warm water and completely dissolved. Then the pH of the solution was adjusted to pH 6.0 with 0.1 M NaOH and 1 ml of a solution of 5 mg peroxidase (Sigma P-6782) that had been previously dissolved in 100 ml of a 100 mM H₂NaPO₄ buffer (pH 6.5) was added. After adding 6 μl of a 3% H₂O₂ solution it was stirred for 10 s, allowed to stand for 1 h at 20° C. and the peroxidase was inactivated by heating the gel for 40 min to 90° C. Subsequently the gel was frozen at −18° C. and dried at 0.1 hPa in the freeze-drying apparatus. Finally 0.3 g of the dried powder was subsequently dissolved in 10 ml warm water at 60° C.

### Example 7

Cross-Linking Plant Extracts with Laccase (Comparison)

3.5 g of a dried sugar beet pulp was added to 100 ml water and the pH of the mixture was adjusted to pH 2 with HNO₃ (28%). Then it was stirred for 15 h at 70° C., the pH was adjusted to a value of 3.3 with 10 N NaOH and it was centrifuged for 20 min at 20° C. and 4000 rpm. Subsequently the supernatant was dialysed:

Dialysis tube Ø 48 mm; 18 ml/cm; MWCO 12,000-14,000; outer phase: 1800 ml water pH 3.5 (adjusted with HNO₃), 20 h; 20° C.; outer phase: 5000 ml water pH 3.5, 22 h; 20° C.

The dialysate was then concentrated at 50° C. and 80 hPa to a dry mass content of 3% before 0.2 mg laccase C (ASA Enzyme, Order No. 2020) dissolved in 1 ml water was added to 25 ml of this solution. It was then allowed to stand for 24 h at 20° C.

### Example 8

Cross-Linking Plant Extracts with Peroxidase

3.5 g dried residues from the sugar beet extraction was added to 100 ml water and the pH of this mixture was adjusted to pH 2.0 with HNO₃ (28%). Then it was stirred for 15 h at 70° C. and the pH was adjusted to pH 3.3 with 10 N NaOH, centrifuged for 20 min at 20° C. and 4000 rpm and the supernatant was concentrated at 50° C. and 80 hPa to a dry mass content of 3%. 0.25 mg peroxidase (Sigma P-6782) dissolved in 1 ml water was added to 25 ml of this solution and 6 μl of a 3% H₂O₂ solution was added and the mixture was allowed to stand to 24 h at 40° C.

### Example 9

Formation of a Gel by Adding an Apple Press Extract to Sugar Beet Pectin

200 g apples were pressed out in a juice extractor (Braun Model 201) and the pressed juice was rotary evaporated under reduced pressure (40 hPa) at 40° C. to a dry mass content of 20%. 10 ml of a 4% sugar beet pectin solution was added to 20 ml of this concentrated pressed juice, it was then stirred and allowed to stand for 24 h at 40° C.

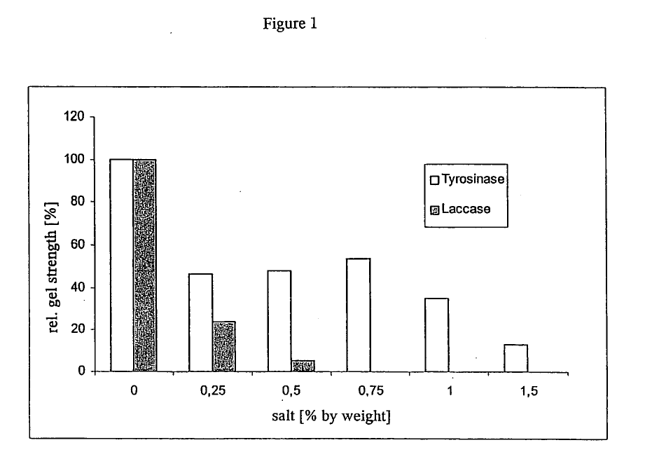

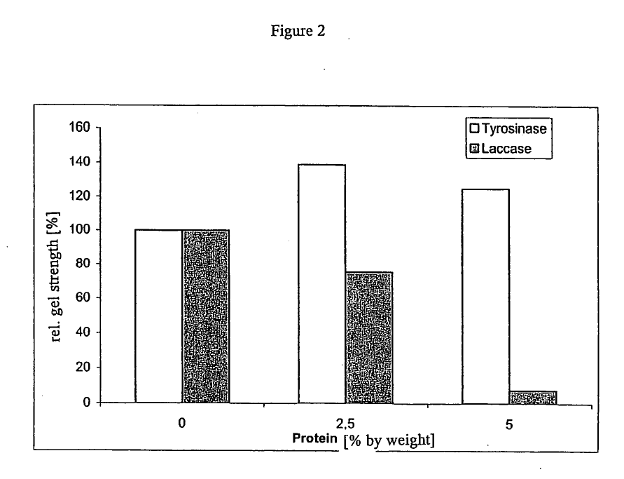

The attached figures further illustrate the present invention. FIGS. 1 and 2 show the different relative gel strengths of reference media from example 4 (laccase) and media according to the invention from example 5 (tyrosinase):

FIG. 1 shows a comparison of the relative gel strengths of tyrosinase and laccase in the presence of various salt concentrations (NaCl).

FIG. 2 shows a comparison of the relative gel strengths of tyrosinase and laccase in the presence of various protein concentrations (bovine serum albumin).

1-23. (canceled)

24. A water-containing medium with increased viscosity comprising water and a gellable polymer component with phenolic substituents modified with the aid of polyphenol oxidases, wherein it is modified by a protein with tyrosinase activity.

25. The medium as claimed in claim 24, wherein it is additionally modified by an enzyme mixture containing hydrolases, oxidoreductases and peroxidases.

26. The medium as claimed in claim 24, wherein it is a gel, optionally in a dried, partially dried, rehydrated or partially rehydrated state.

27. The medium as claimed in claim 24, wherein the polymer component carries monophenolic substituents.

28. The medium as claimed in claim 24, wherein the polymer component is at least one polysaccharide and in particular one containing (un)substituted cinnamic ester groups.

29. The medium as claimed in claim 28, wherein the at least one polysaccharide is arabinoxylan or a pectin.

30. The medium as claimed in claim 29, wherein the medium contains pectin that is derived from Chenopodiaceae, sugar beet or pulps thereof.

31. The medium as claimed in claim 29, wherein the medium contains pectin from which at least one of the arabinose groups was removed, optionally under slightly acidic conditions at a pH between 6.0 and 7.5 or with the aid of an enzyme or arabinofuranosidase.

32. The medium as claimed in claim 30, wherein the arabinoxylan component is derived from a cereal, maize, wheat, flour or coarse meal.

33. The medium as claimed in claim 24, wherein the polymer was modified by tyrosinase [EC 1.14.18.1].

34. The medium as claimed in claim 24, wherein the protein with tyrosinase activity is derived from a plant of the Solanaceae family, potatoes, apples, aubergines, chicory, bananas, avocados, tea plants or mushrooms.

35. The medium as claimed in claim 24, wherein the modification was carried out using an enzyme mixture containing a β-galactosidase, glucose oxidase, peroxidase and optionally a catalase.

36. The medium as claimed in claim 24, wherein the medium is dried.

37. The medium as claimed in claim 24, wherein the enzyme responsible for the modification are present in an inactive form after the modification is completed.

38. The medium as claimed in claim 37, wherein the medium contains enzymes that have been chemically or thermally inactivated.

39. A composition comprising a pharmaceutical adjuvant, cosmetic adjuvant, food or texturing agent and the medium of claim 24.

40. A process for producing a water-containing medium having an increased viscosity, comprising
a) dissolving at least a portion of a gellable polymer component containing phenolic substituents in an aqueous medium, subsequently
b) adding at least one of a partly dissolved oxidoreductase, peroxidase, hydrolase or catalase of plant or fungal origin at room temperature to the solution from step a), and subsequently
c) stirring the solution from step b) for at least 15 minutes at temperatures between 15 and 60° C., and
d) thermally, chemically, or thermally and chemically inactivating the enzymes present in the solution obtained from step c).

41. A process as claimed in claim 40, wherein in process step a) at least one member from selected from the group consisting of oligosaccharide- or polysaccharide-, alcohol-, lactate-, glutamate-, pectin- and a lactose-containing medium, preferably milk or a milk-containing medium is added first as a polymer component.

42. A process as claimed in claim 40, wherein at least one galactosidase, glucose oxidase, horseradish peroxidase, laccase or polyphenol oxidase is added in process step b).

43. A process as claimed in claim 40, wherein the solution obtained from process step b) is subjected to at least one drying step after optionally adding further gellable and optionally modified polymers.

44. A process as claimed in claim 43, wherein the powder obtained from the drying step is rehydrated.

45. The medium produced by the process of claim 40.

46. The medium as claimed in claim 45, wherein the enzymes added in process step b) are present in the medium in an inactive form.

47. A composition comprising a pharmaceutical adjuvant, cosmetic adjuvant, food or texturing agent and the medium of claim 40.
